(19) United States
(12) Patent Application Publication
(10) Pub. No.: US 2026/0301865 A1
(43) Pub. Date: Oct. 1, 2026

(54) EXPERT PANEL SUPPORT SYSTEM AND EXPERT PANEL SUPPORT METHOD

(71) Applicant: HITACHI HIGH-TECH CORPORATION, Tokyo (JP)

(72) Inventors: Eiji KANAMORI, Tokyo (JP); Koichiro KIMOTSUKI, Tokyo (JP); Aya KURIKI, Tokyo (JP); Eichi WATABE, Tokyo (JP)

(73) Assignee: HITACHI HIGH-TECH CORPORATION, Tokyo (JP)

(21) Appl. No.: 19/574,680

(22) Filed: Mar. 23, 2026

(30) Foreign Application Priority Data
Mar. 27, 2025 (JP) 2025-054141

(51) Int. Cl. G16B 20/20; G16B 45/00; G16H 40/63

(52) U.S. Cl. CPC G16B 20/20; G16B 45/00; G16H 40/63

(57) ABSTRACT

Provided is an expert panel support system for displaying a test result of a patient subjected to a gene panel test which is a target of an expert panel. The expert panel support system includes an extraction unit configured to extract information related to a gene variant of the patient in a plurality of the gene panel tests, and a display processing unit configured to display a change in the gene variant on a terminal used by a member of the expert panel in a form visually recognizable by the member.

### BACKGROUND OF THE INVENTION

### 1. Field of the Invention

The present invention relates to a technique of an expert panel support system and an expert panel support method.

### 2. Description of Related Art

Cancer genome medical care has been performed as daily care. In the cancer genome medical care, a cancer gene panel test in which several tens to several hundreds of cancer genes are tested at a time is performed for one patient. A change in a cancer gene is confirmed according to a test result of the cancer gene panel test.

An expert panel, which is a meeting for performing medical interpretation based on a result of such a cancer gene panel test and studying an optimal treatment policy, is held. In the expert panel (expert meeting), an expert such as a doctor specializing in cancer medicine therapy or a doctor specializing in genetic medicine discusses a treatment policy based on the test result of the cancer gene panel test.

PTL 1 discloses a report creation method and a report creation device for creating a report for reporting a level of pathogenicity of a gene variant, the report creation method including: acquiring information on a gene variant detected in a gene panel test; creating an evaluation result for a plurality of evaluation items based on a first criterion for evaluating each of the evaluation items and at least the information on the gene variant; determining a level of pathogenicity based on a second criterion for determining the level of pathogenicity and the evaluation result; determining an undetermined item for which the evaluation result is undetermined among the plurality of evaluation items; determining whether the level of the pathogenicity is provisional based on a determination result of the undetermined item; and creating a report for reporting the level of pathogenicity according to the determination result of whether the level of pathogenicity is provisional.

### Citation List

### Patent Literature

PTL 1: JP2023-080642A

### SUMMARY OF THE INVENTION

The role required of an expert panel in the future is to monitor a treatment progress and study an optimal treatment by applying a gene panel test to blood cancer or using the gene panel test from an initial treatment even for solid cancer. Therefore, it is required to perform a test a plurality of times on the same case. However, at present, a report of a test company and information of an expert panel preliminary study are not systematically integrated, and it is difficult to easily grasp and study a case.

In blood cancer or the like, a gene panel test is performed a plurality of times. Therefore, a load on gene variant interpretation is expected to increase.

In a cancer gene panel test, one patient may be subjected to a plurality of tests, but PTL 1 does not describe that a plurality of cancer gene panel tests are displayed.

The invention has been made in view of such a background, and an object of the invention is to allow a change in results of a plurality of cancer gene panel tests to be visually recognized easily.

In order to solve the above-described problem, the invention provides an expert panel support system for displaying a test result of a patient subjected to a gene panel test which is a target of an expert panel, the expert panel support system including: an extraction unit configured to extract information related to a gene variant of the patient in a plurality of the gene panel tests; and a display processing unit configured to display a change in the gene variant on a terminal used by a member of the expert panel in a form visually recognizable by the member.

Other solutions will be described as appropriate in the embodiments.

According to the invention, a change in results of a plurality of cancer gene panel tests can be visually recognized easily.

### BRIEF DESCRIPTION OF THE DRAWINGS

FIG. 1 is a diagram showing a configuration of an expert panel support system according to an embodiment.

FIG. 2 is a diagram showing a hardware structure of a processing device according to the present embodiment.

FIG. 3 is a diagram showing a configuration of registration information.

FIG. 4 is a diagram showing an example of focused variant information stored in a focused variant DB.

FIG. 5 is a diagram showing an example of user related information.

FIG. 6 is a flowchart showing an overall procedure of an expert panel support method according to the embodiment.

FIG. 7 is a flowchart showing a procedure of standardization processing executed by a conversion unit.

FIG. 8A is a flowchart showing a procedure of an expert panel support screen creation processing executed by a processing device.

FIG. 8B is a flowchart showing a procedure of the expert panel support screen creation processing executed by the processing device.

FIG. 9 is a diagram showing an example of an expert panel support screen.

### DESCRIPTION OF EMBODIMENTS

Next, modes for implementing the invention (referred to as "embodiments") will be described in detail with reference to the drawings as appropriate.

In the embodiment, a genome and a gene are distinguished as follows. (1) Gene: a range of DNA that produces (translates) a protein. (2) Genome: all genes in a certain organism.

In the present embodiment, a gene panel test is appropriately referred to as a "test". A "variant" of a gene variant refers to a variant as compared with a healthy person.

### System Configuration

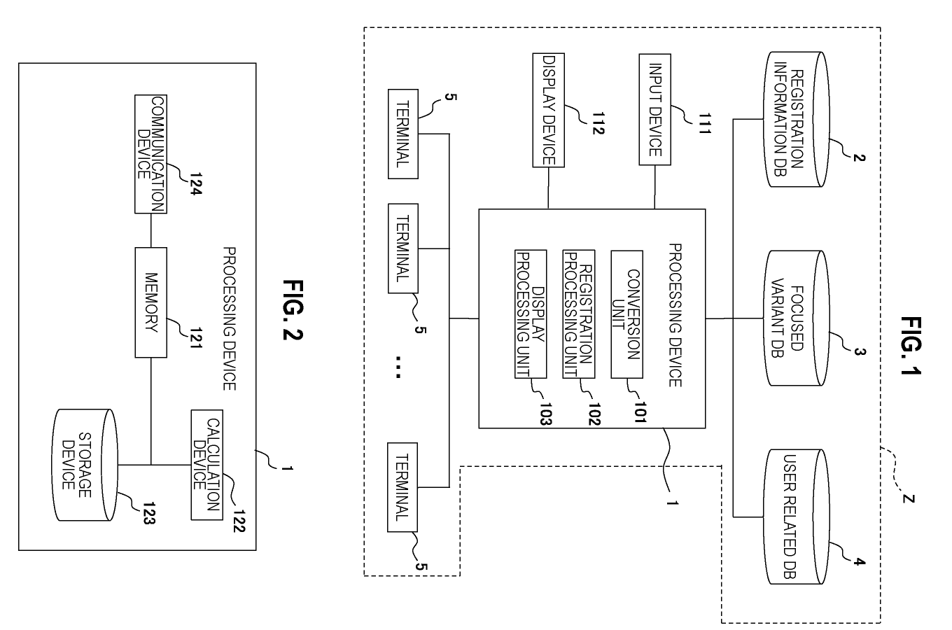

FIG. 1 is a diagram showing a configuration of an expert panel support system Z according to the present embodiment.

The expert panel support system Z displays a test result of a patient subjected to a gene panel test which is a target of an expert panel. The expert panel support system Z includes a processing device 1, a registration information DB 2, a focused variant DB 3, a user related DB 4, and terminals 5 used by members (panelist of an expert panel) who perform expert panel preliminary studies. The member is a member of the expert panel. A member who uses the terminal 5 may be referred to as a "user".

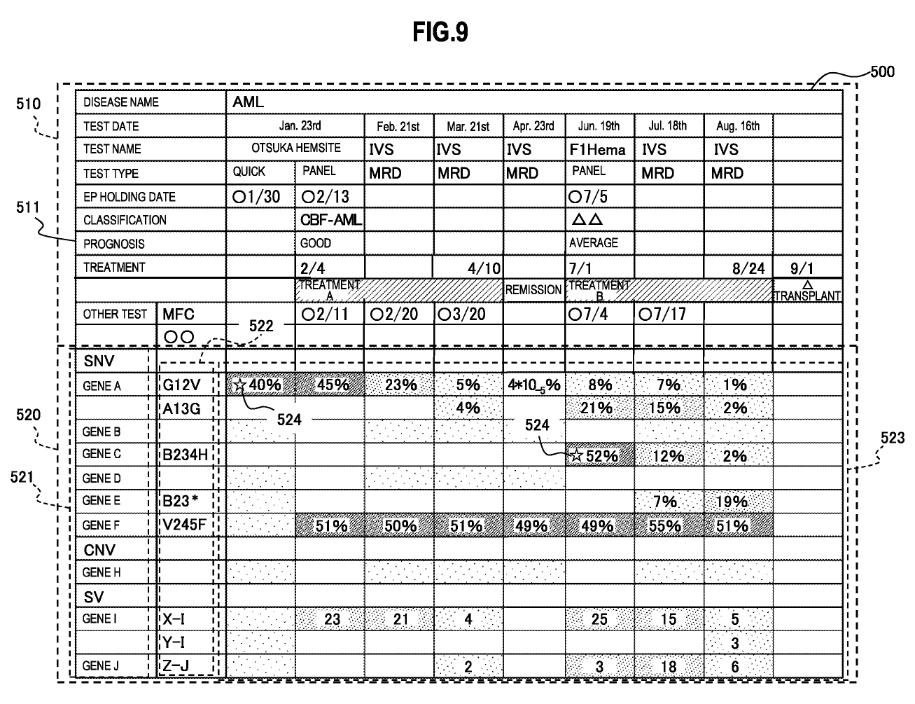

The processing device 1 creates an expert panel support screen 500 shown in FIG. 9 and causes the terminal 5 to display the created expert panel support screen 500.

The processing device 1 includes a conversion unit 101, a registration processing unit 102, and a display processing unit 103.

The conversion unit 101 converts gene information that varies depending on a test into standard information. The registration processing unit 102 registers the information in the registration information DB 2. The display processing unit 103 displays a change in a gene variant on the terminal 5 used by a member in a form visually recognizable by the members of the expert panel. The form in which the member of the expert panel can visually recognize the change in the gene variant is displayed on the terminal 5 as the expert panel support screen 500 shown in FIG. 9. The display processing unit 103 is also an "extraction unit" or an "acquisition unit". The change in the gene variant is a change in a level of a variant (variant level) in the gene variant. In the present embodiment, a change in a value of the variant level over time is referred to as a gene variant change.

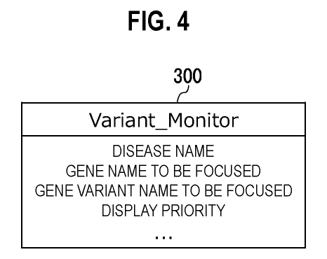

The registration information DB 2 stores registration information 200 (see FIG. 3) including information related to a patient, information related to a test, and information related to a gene variant. The registration information 200 will be described later.

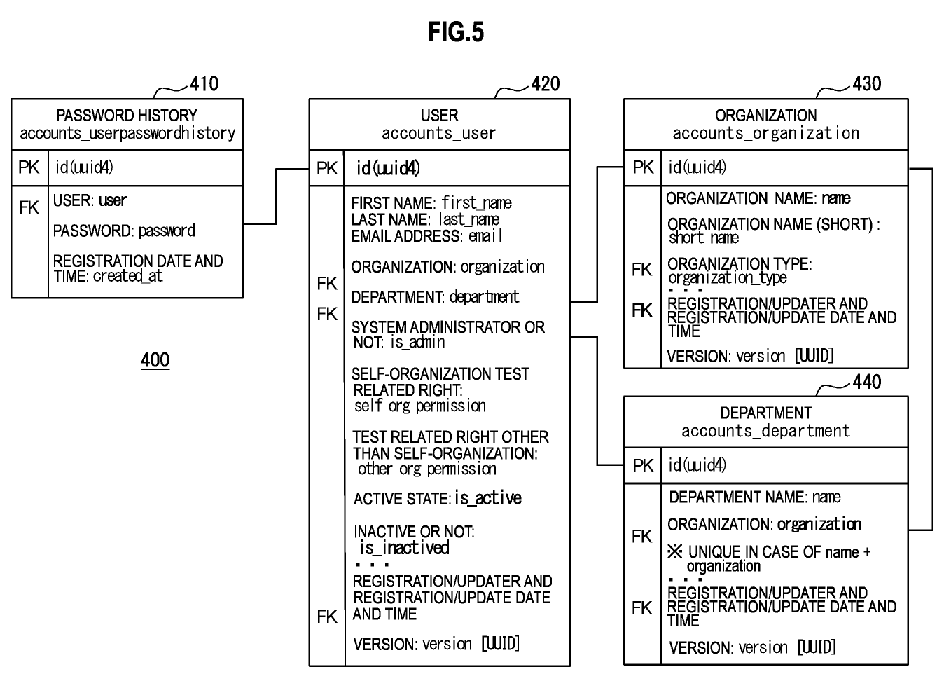

Focused variant information 300 (see FIG. 4) is stored in the focused variant DB 3. The focused variant information 300 stores information related to a variant to be focused for a disease and a priority for display (display priority). The focused variant information 300 will be described later.

The user related DB 4 stores user related information 400 in which an access right and the like of a corresponding user are registered. The user related information 400 will be described later.

The processing device 1 is provided with an input device 111 such as a keyboard or a mouse, and a display device 112 such as a display.

A medical institution server 6 is a computer installed in a medical institution, and transmits information such as a test result of the gene panel test to the processing device 1.

### Hardware structure

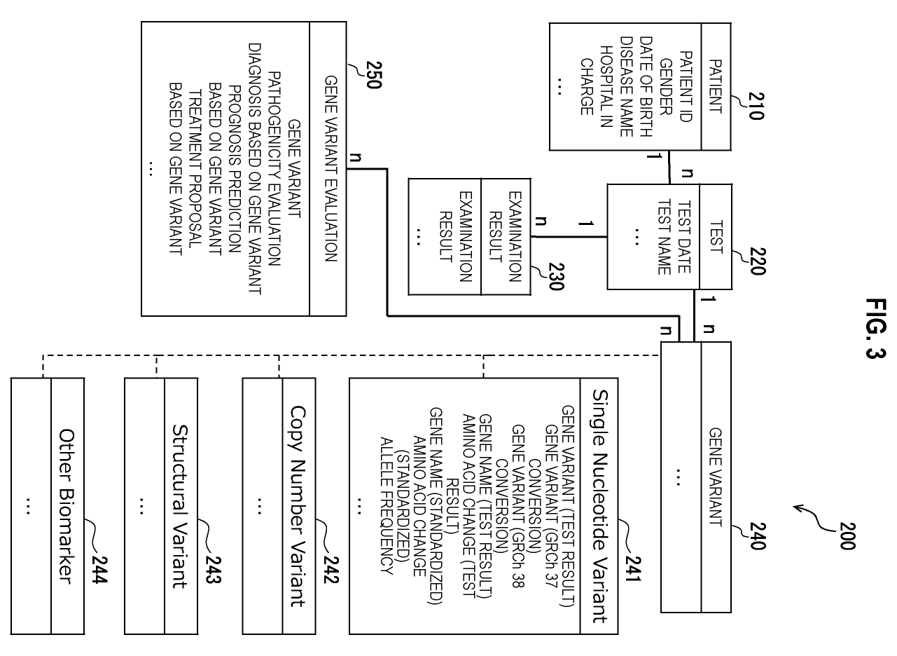

FIG. 2 is a diagram showing a hardware structure of the processing device 1 according to the present embodiment.

The processing device 1 is implemented by a personal computer (PC) or the like, and includes a memory 121, a calculation device 122, a storage device 123, and a communication device 124.

The memory 121 is implemented by a random access memory (RAM) or the like. The calculation device 122 is implemented by a central processing unit (CPU), a graphic processing unit (GPU), and the like. The storage device 123 is implemented by a hard disk drive (HDD), a solid state drive (SSD), and the like. Then, the communication device 124 communicates with each of the terminals 5, the medical institution server 6, the registration information DB 2, the focused variant DB 3, and the user related DB 4.

A program stored in the storage device 123 is loaded into the memory 121. Then, the loaded program is executed by the calculation device 122 to implement functions of the conversion unit 101, the registration processing unit 102, and the display processing unit 103 shown in FIG. 1.

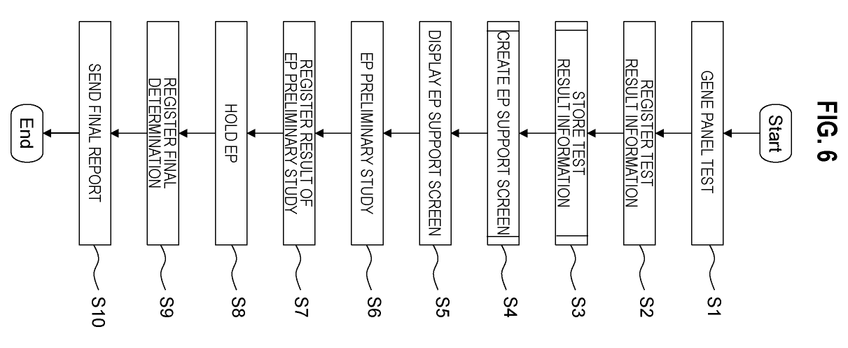

In the example shown in FIG. 1, it is assumed that each of the registration information DB 2, the focused variant DB 3, and the user related DB 4 is managed by a database server (not shown) different from the processing device 1. However, the invention is not limited thereto, and any one of the focused variant information 300 (see FIG. 3), the focused variant information 300 (see FIG. 4), and the user related information 400 (FIG. 5) may be stored in the storage device 123 of the processing device 1.

### Registration Information 200

FIG. 3 is a diagram showing a configuration of the registration information 200.

As shown in FIG. 3, the registration information 200 includes patient information 210, test information 220, examination result information 230, gene variant information 240, and evaluation information 250. The patient information 210, the test information 220, the examination result information 230, the gene variant information 240, and the evaluation information 250 are associated with one another in the form of a relational database.

The patient information 210 stores information such as a "patient ID", "gender", "date of birth", a "disease name", and a "hospital in charge".

The test information 220 stores information such as a "test date" and a "test name".

"1" and "n" in FIG. 3 indicate that one piece of information denoted by "1" corresponds to a plurality of pieces of information denoted by "n". For example, in FIG. 3, "1" is written for the patient information 210 and "n" is written for the test information 220, and one piece of the patient information 210 corresponds to a plurality of pieces of the test information 220. That is, in this example, one patient is subjected to a plurality of tests (gene panel tests).

The test information 220 corresponds to a plurality of pieces of the examination result information 230. The examination result information 230 stores an examination result of an expert panel.

The test information 220 is stored corresponding to a plurality of pieces of the gene variant information 240. That is, the gene variant information 240 stores information related to a test.

The gene variant information 240 includes a plurality of pieces of sub-information. The sub-information includes single nucleotide variant information (SNV) 241 and copy number variant information (CNV) 242. Further, the sub-information of the gene variant information 240 includes structural variant information (SV) 243 and other biomarker information (other biomarker) 244.

The single nucleotide variant information 241 mainly stores information on a single nucleotide variant. Information stored in the single nucleotide variant information 241 includes a gene variant (test result), a gene variant (GRCh37 conversion), a gene variant (GRCh38 conversion), and the like. The information stored in the single nucleotide variant information 241 further includes a gene name (test result), an amino acid change (test result), a gene name (standardized), an amino acid change (standardized), an allele frequency, and the like.

In the above description, a "test result" is described in test result information, and "standardized" indicates that a test result is standardized.

Although there are international standard names (standard information) for gene information such as a gene variant name or a name of a gene in which a gene variant occurred, a name or information different from the standard may be used for the following reason or the like.

### 1 Differences in Reference Genomes

A position of a reference genome may be updated when the genome is re-read. For example, in the past, "GRCh37" was used as a reference genome and a genome position was "12: 25398234", but the genome position in the latest reference sequence "GRCh38" is "12: 25245350". "GRCh" indicates an "international human genome reference sequence (Genome Research Consortium human)", and "37" and "38" indicate versions. "12" indicates a chromosome number, and "25398234" and "25245350" indicate positions of genes (genomes).

That is, positions of substantially identical gene variants are described as "25398234" or "25245350" depending on versions. In a test result, a position of a gene may be described in an old version. However, it may be necessary to use the latest version in an expert panel in the present embodiment. In such a case, the conversion unit 101 converts a position of a gene described as "GRCh37 12: 25398234" into "GRCh38 12: 25245350".

### 2 Difference in Gene Names

For example, "CD274" and "PD-L1" indicate the same gene. "CD274" indicates a gene name, and "PD-L1" indicates a protein expressed by "CD274". A gene name is used, or a protein expressed by a gene is commonly used as an alias of the gene name depending on a test institution. Therefore, when "PD-L1" is used as a test result, it is necessary to convert "PD-L1" into "CD274" to unify the gene name.

The same applies to "ERBB2" and "HER2". In this case, "ERBB2" is a gene name, and "HER2" indicates a protein expressed by "ERBB2". In this case, it is necessary to convert "HER2" into "ERBB2" as a test result.

### 3Differences in Transcript Definition of Protein Range

A transcript (definition of protein range) may differ depending on a test institution. "NP_001341538.1:p.Val600Glu" and "NP_001361173.1:p.Val640Glu" indicate protein variants derived from the same gene variant. A difference between these two notations is a difference depending on which transcription product (protein) is used as a reference. The conversion unit 101 unifies such differences in transcripts.

### 4 Difference in Simple Notations

Even the same gene variant may have different notations depending on a medical institution. For example, a certain gene variant may be described as "p.Gly12Asp" in a certain medical institution, but may be denoted as "p.G12D" in another medical institution. Thus, when a notation of a gene variant differs depending on a medical institution, the notation needs to be unified.

As described above, even substantially identical gene variants may have different notations such as gene names, gene positions, and the like depending on tests. In order to unify such different notations, the conversion unit 101 converts information on a gene variant described in a test result into information on a gene variant based on another reference sequence. The other reference sequence is information on a gene having a version serving as a criterion for standardization. Such processing is referred to as standardization.

A "gene variant (test result)" may be "GRCh37" or "GRCh38" depending on tests. Therefore, the converted "gene variant (GRCh37 conversion)" and "gene variant (GRCh38 conversion)" are stored together.

An allele frequency is stored in test result information sent from a medical institution.

Information stored in the copy number variant information 242, the structural variant information 243, and the other biomarker information 244 is substantially the same as the information stored in the single nucleotide variant information 241. However, the copy number variant information 242 stores a copy number instead of an allele frequency, and the structural variant information 243 stores a read number instead of an allele frequency.

A plurality of pieces of the gene variant information 240 are associated with a plurality of pieces of the evaluation information 250.

The evaluation information 250 stores information on evaluation of a gene variant evaluated by an expert panel and a diagnosis performed by a doctor. Specifically, the evaluation information 250 stores information related to pathogenicity evaluation of a gene variant, a diagnosis based on a gene variant, prognosis prediction based on a gene variant, and a treatment proposal based on a gene variant. In the present embodiment, the prognosis prediction based on a gene variant is prognosis predicted according to a gene variant detected as a result of a cancer gene panel test. Prognosis prediction includes "good", "average", "poor", and the like.

The registration information 200 may be not in the form of a relational database.

### Focused Variant Information 300

FIG. 4 is a diagram showing an example of the focused variant information 300 (variant monitor) stored in the focused variant DB 3.

As described above, the focused variant information 300 stores information related to a variant to be focused for a disease and a priority for display (display priority). Specifically, a disease name, a gene name to be focused, a gene variant name to be focused, and a display priority are stored.

The display priority is a priority order related to a disease of a patient. Depending on the display priority, information related to a gene variant to be focused in a disease is preferentially displayed. A gene variant name to be focused is a gene variant name necessary for disease type classification or prognosis prediction, which is a medicine target (treatment target).

In the case of acute myeloid leukemia (AML), examples of gene variants to be focused on in the disease include "RUNX1::RUNX1T1", "CBFB::MYH11", and "WT". The focused variant information 300 is registered in advance before a test is performed on a patient.

### User Related Information 400

FIG. 5 is a diagram showing an example of the user related information 400.

The user related information 400 includes password history (accounts_userpasswordhistory) information 410 and user (accounts_user) information 420. Further, the user related information 400 includes organization (accounts_organization) information 430 and department (accounts_department) information 440.

The password history information 410, the user information 420, the organization information 430, and the department information 440 have a relational database format associated by a primary key (PK). In the example shown in FIG. 5, the primary key is created in the format of uuid4, but may be created in a format other than uuid4.

The password history information 410 stores information on a "user", a "password", and a "registration date and time (created_at)" of the password. In the example shown in FIG. 5, information on a "user" is set as an external key (FK).

The user information 420 stores information such as "first_name", "last_name", and "email address". Further, the user information 420 stores information such as "organization", "department", and "whether a user is a system administrator (is_admin)". In addition, the user information 420 stores information such as "self-organization test related right (self_org_permission)" and "test related right other than self-organization (other_org_permission)". The user information 420 stores information related to an "active state (is_active)" and "whether an account is inactive (is_inactived)". Further, the user information 420 stores information such as "registration/updater and registration/update date and time" and "version". In the example shown in FIG. 5, the information of "version" is stored in the UUID format, but may not be stored in the UUID format.

In the example shown in FIG. 5, among the information constituting the user information 420, "organization", "department", and "registration/updater and registration/update date and time" are set as external keys.

An access right of a user to test related information is set by "self-organization test related information" and "test related information other than self-organization". Accordingly, setting is performed regarding whether a user can access only self-organization test related information or can access other test related information.

The organization information 430 stores information such as an "organization name (name)", an "organization name (short) (short_name)", and an "organization type (organization_type)". The organization information 430 stores information such as "registration/updater and registration/update date and time" and "version". In the example shown in FIG. 5, the information of "version" is stored in the UUID format, but may not be stored in the UUID format.

In the example shown in FIG. 5, an "organization type" and "registration/updater and registration update date and time" are set as an external key (FK).

The department information 440 stores information such as a "department name (name)" and an "organization". The organization information 430 stores information such as "registration/updater and registration/update date and time" and "version". In the example shown in FIG. 5, the information of "version" is stored in the UUID format, but may not be stored in the UUID format.

In the example shown in FIG. 5, an "organization type" and "registration/updater and registration update date and time" are set as an external key (FK).

In addition, "department name" + "organization" is unique information.

### Flowchart

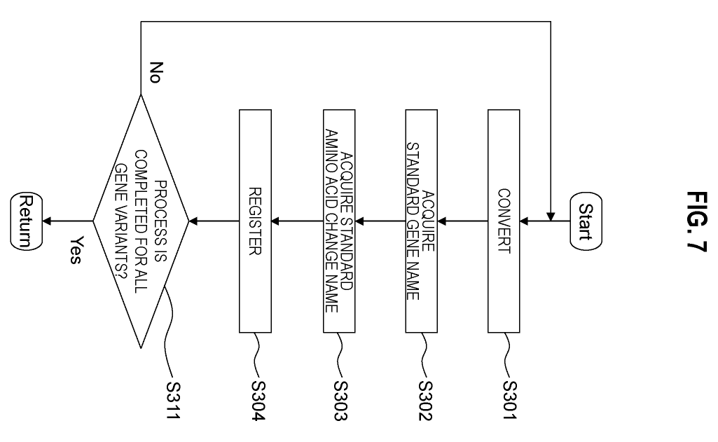

Next, an expert panel support method according to the present embodiment will be described with reference to FIGS. 6 to 8B.

### Overall Processing

FIG. 6 is a flowchart showing an overall procedure of the expert panel support method according to the present embodiment. Reference is made to FIG. 1 as appropriate.

First, a gene panel test is performed on a patient (S1). The test (gene panel test) may be performed on one patient for a plurality of times.

Then, test result information of the test is registered in the processing device 1 using a medical terminal (not shown) installed in a medical institution (S2). As described above, since the test may be performed a plurality of times, the test result information registered in step S2 is information on test results of a gene panel test performed a plurality of times. The test result information registered in step S2 is test result information on one patient. The test result information is generally in a file format such as an xml file or a json file, but may be in other file formats.

Subsequently, the conversion unit 101 executes standardization processing for a gene name, a gene variant name, and the like, and the registration processing unit 102 stores the test result information registered in step S2 and a result of the standardization processing in the registration information 200 (S3). Details of the processing in step S3 will be described later.

Since the processing in steps S1 to S3 are executed each time the test is performed, the processing is generally performed a plurality of times.

A specific procedure of the standardization processing for the gene name, the gene variant name, and the like will be described later.

Subsequently, when an expert panel preliminary study is performed, the display processing unit 103 creates an expert panel (EP) support screen for displaying variant levels in time series (S4). Then, the display processing unit 103 displays the created expert panel (EP) support screen on the terminal 5 used by a member who performs the expert panel preliminary study (S5). Details of the processing in step S4 will be described later. Step S5 is a "display processing step".

The variant level indicates an abnormality level of a gene variant. Examples of the variant level include an allele frequency, a copy number of a gene in a copy number variant, and a read number (expression level).

The expert panel preliminary study is performed in advance by each member who participates in an expert panel before the expert panel is held. In the expert panel preliminary study, each member considers a recommended treatment proposal. At this time, each member considers a recommended treatment proposal based on a result of a previous test, a result of an expert panel held in the past, a result of a current test, and the like.

Each member performs an expert panel (EP) preliminary study based on the expert panel support screen 500 (see FIG. 9) displayed in step S4 (S6).

Then, each member who has performed the expert panel preliminary study registers a result of the expert panel (EP) preliminary study in the registration information DB 2 via the used terminal 5 (S7). The registered information on the result of the expert panel preliminary study is registered in the examination result information 230 shown in FIG. 3.

Then, the expert panel (EP) is held (S8). In the expert panel, members of the expert panel discuss a treatment policy, a recommended medicine, the necessity of clinical trial, and the like based on results of the expert panel preliminary study and the like. In the expert panel, a screen similar to the expert panel support screen 500 may be displayed on the terminal 5, or other screens may be displayed on the terminal 5.

When the expert panel ends, a member of the expert panel registers a final determination of the expert panel (S9). The registration is performed via the terminal 5 used by the member of the expert panel. The registered information is stored in the examination result information 230 in FIG. 3. The registered information includes a treatment policy, a recommended medicine, necessity of a clinical trial, and the like.

The processing device 1 creates a final report based on the information registered in step S9, and sends the created final report to a doctor in charge (S10).

### Standardization Processing

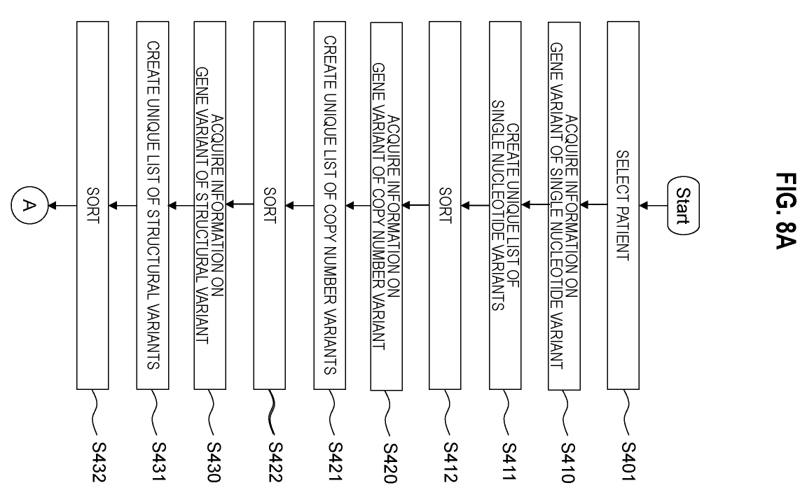

FIG. 7 is a flowchart showing a procedure of the standardization processing executed by the conversion unit 101. In the processing shown in FIG. 7, the processing executed by the conversion unit 101 in the processing of step S3 in FIG. 6 will be described. Reference is made to FIGS. 1 and 3 as appropriate.

First, the conversion unit 101 converts a gene variant name included in the test result information of the gene panel test into a gene variant name in another reference sequence to convert the gene variant name into a standardized gene variant name (standard gene variant name) (S301). The other reference sequence includes a reference sequence of a different version. The processing of step S301 is executed by the conversion unit 101 referring to a database (not shown) or the like installed outside the expert panel support system Z. Hereinafter, a database installed outside the expert panel support system Z is referred to as an external database.

There is a case where no target standard gene variant name is present in a database to be referred to. In such a case, a user manually convert the gene variant name into a standard gene variant name via the input device 111. The same applies to an amino acid change name.

Subsequently, the conversion unit 101 acquires a standardized gene name (standard gene name) using the gene variant in the reference sequence converted in step S301 as a key (S302). In step S302, the conversion unit 101 searches an external database (not shown) or the like.

Then, the conversion unit 101 acquires a standardized amino acid change name (standard amino acid change name) using the gene variant in the reference sequence converted in step S301 as a key (S303). In step S303, the conversion unit 101 searches an external database (not shown) or the like.

Thereafter, the registration processing unit 102 registers the converted standard gene variant name, the acquired standard gene name, and the acquired standard amino acid change name in the gene variant information 240 (S304). At this time, the registration processing unit 102 registers the acquired standard gene variant name, the acquired standard gene name, and the acquired standard amino acid change name in information including the single nucleotide variant information 241 to the other biomarker information 244 depending on a type of the gene variant. The type of the gene variant is acquired by the registration processing unit 102 searching an external database (not shown) or the like using the standard gene name or the like as a key. Alternatively, the type of the gene variant may be described in the test result information in advance. The standard gene variant names correspond to the "gene variant (GRCh37 conversion)" and the "gene variant (GRCh38 conversion)" in FIG. 3. The standard gene name corresponds to the "gene name (standardized)" in FIG. 3, and the standard amino acid change name corresponds to the "amino acid change (standardized)" in FIG. 3.

Then, the conversion unit 101 determines whether the processing of steps S301 to S304 is completed for all gene variants described in the test result (S311).

When the processing of steps S301 to S304 is not executed for all the gene variants described in the test result (S311: No), the conversion unit 101 executes the processing in step S301 for a new gene variant.

When the processing of steps S301 to S304 is completed for all the gene variants described in the test result (S311: Yes), the processing device 1 returns the processing to step S4 in FIG. 6.

The conversion unit 101 converts information that indicates substantially identical gene variants but is different between gene panel tests into unified information by standardization by the processing shown in FIG. 7.

The "gene variant (test result)" in the single nucleotide variant information 241 in FIG. 3 is converted into the "gene variant (GRCh37 conversion)" and the "gene variant (GRCh38 conversion)" by the processing shown in FIG. 7. Similarly, the "gene name (test result)" is converted into the "gene name (standardized)", and the "amino acid change (test result)" is converted into the "amino acid change (standardized)".

### Processing of Creating Expert Panel Support Screen 500

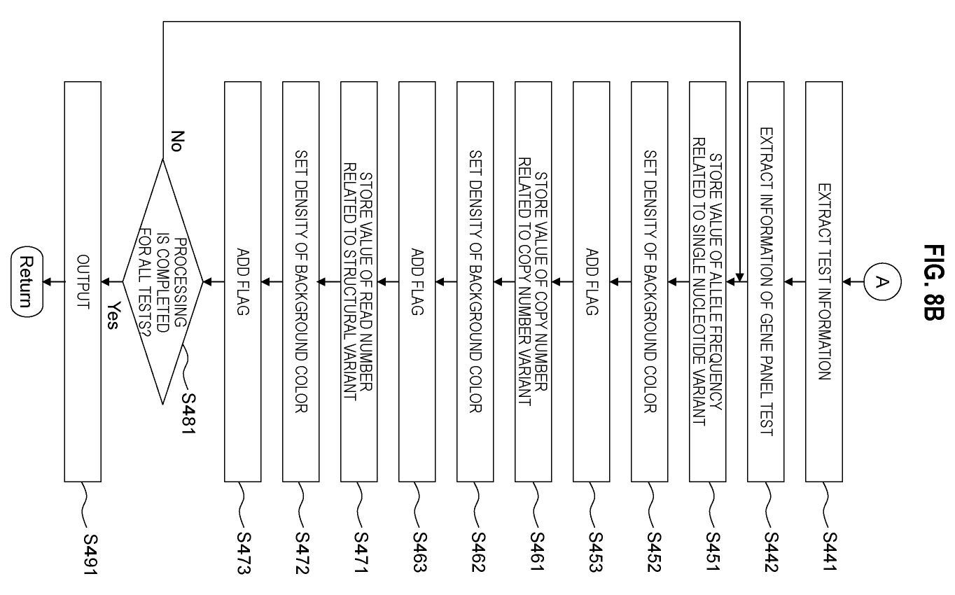

FIGS. 8A and 8B are flowcharts showing a procedure of processing of creating the expert panel support screen 500 executed by the processing device 1. The processing shown in FIGS. 8A and 8B is processing executed in step S4 of FIG. 6, and is processing executed by the display processing unit 103 of the processing device 1. Reference is made to FIGS. 1 and 3 as appropriate. FIGS. 8A and 8B show processing related to creation of a part of a variant information time series field 523 on the expert panel support screen 500 shown in FIG. 9.

First, a user selects a patient to be tested via the terminal 5 (S401 in FIG. 8A).

Subsequently, the display processing unit 103 acquires, from the single nucleotide variant information 241, information on gene variants for all single nucleotide variants associated with all tests of the selected patient (S410). A set of a standard gene variant name and a standard amino acid change name is obtained. The standard gene variant name corresponds to the "gene variant name (GRCh38 conversion)" or the like shown in FIG. 3. Similarly, the standard amino acid change name corresponds to the "amino acid change (standardized)" shown in FIG. 3.

Then, the registration processing unit 102 creates a unique list of single nucleotide variants using the set of the standard gene variant name and the standard amino acid change name (S411). The unique list refers to a list unique to each patient.

Then, the display processing unit 103 sorts the list obtained in step S411 (S412). In the processing of step S412, the display processing unit 103 sorts gene variants in descending order of priority in the focused variant information 300. A sorting result is referred to as a "single nucleotide variant (SNV) display order".

Subsequently, the display processing unit 103 acquires, from the copy number variant information 242, information on gene variants for all copy number variants associated with all tests of the selected patient (S420). Then, the display processing unit 103 creates a unique list of standard gene variant names for the acquired copy number variant information (S421). The acquired gene variant information is a set of a standard gene variant name and a standard amino acid change name.

Then, the display processing unit 103 sorts the list obtained in step S421 (S422). In the processing of step S422, the display processing unit 103 sorts gene variants in descending order of priority in the focused variant information 300. A sorting result is referred to as a "copy number variant (CNV) display order".

Next, the display processing unit 103 acquires, from the structural variant information 243, gene variant information for all structural variants associated with all tests for the selected patient (S430). Then, the display processing unit 103 creates a unique list of standardized structural variants (S431). The acquired gene variant information is a set of a standard gene variant name and a standard amino acid change name.

Then, the display processing unit 103 sorts the list obtained in step S431 (S432). In the processing of step S432, the display processing unit 103 sorts the variants in descending order of priority in the focused variant information 300. A sorting result is referred to as a "structural variant (SV) display order".

The processing of steps S410, S420, and S430 is an extraction step of extracting information related to gene variants of the patient in a plurality of gene panel tests. In steps S412, S422, and S432, the display processing unit 103 executes processing of displaying variant levels of gene variants based on a priority order related to a disease of the patient for the gene variants presented to a member.

Next, the display processing unit 103 extracts information (test information 220) of a test for which the processing of steps S451 to S456 to be described below is not executed among tests performed on the patient who is a processing target (S441 in FIG. 8B). Then, the display processing unit 103 extracts information of a test (gene panel test) with an oldest date from the extracted test information 220 (S442). Step S442 is executed by the display processing unit 103 referring to the test information 220 shown in FIG. 3. In steps S441 and S442, the examination result information 230, the gene variant information 240, the evaluation information 250, and the like associated with the extracted test information 220 are also extracted. The gene variant information 240 includes the single nucleotide variant information 241, the copy number variant information 242, the structural variant information 243, and the like. Accordingly, the display processing unit 103 acquires a variant level of a gene variant for each gene panel test in steps S441 and S442.

Subsequently, the display processing unit 103 stores a value of an allele frequency related to a single nucleotide variant according to the "SNV display order" in an array (S451). The allele frequency is included in a test result.

Then, the display processing unit 103 sets a density of a background color according to the value of the allele frequency (S452). The background color is a color of a field in which the allele frequency is displayed on the expert panel support screen 500 shown in FIG. 9. when no single nucleotide variant is detected in a test according to the "SNV display order", the display processing unit 103 sets a blank.

Subsequently, when a single nucleotide variant to be processed is a treatment target gene variant which is an examination result of a test, the display processing unit 103 adds a flag (S453). This flag is used for displaying a star mark 524 in the variant information time series field 523 shown in FIG. 9. The treatment target gene variant is a gene variant which is a treatment target. Whether a gene variant is a treatment target gene variant is determined by the display processing unit 103 determining whether the gene variant is registered as a gene variant to be focused for a disease of a patient in the focused variant information 300 shown in FIG. 4.

Thereafter, the display processing unit 103 stores a value of a copy number of a copy number variant according to the "CNV display order" in an array (S461). The copy number is included in a test result.

Then, the display processing unit 103 sets a density of a background color according to the value of the copy number (S462). When no copy number variant is detected in a test according to the "CNV display order", the display processing unit 103 sets a blank.

Subsequently, when the copy number variant to be processed is a treatment target gene variant which is an examination result of a test, the display processing unit 103 adds a flag (S463). This flag is for displaying the star mark 524 in the variant information time series field 523 shown in FIG. 9.

Subsequently, the display processing unit 103 stores a value of a read number of a structural variant according to the "SV display order" in an array (S471). The read number is included in a test result.

Then, the display processing unit 103 sets a density of a background color according to the value of the read number (S472). When no structural variant is detected in a test according to the "SV display order", the display processing unit 103 sets a blank.

Subsequently, when a structural variant to be processed is a treatment target gene variant which is an examination result of a test, the display processing unit 103 adds a flag (S473). This flag is for displaying the star mark 524 in the variant information time series field 523 shown in FIG. 9.

Next, the display processing unit 103 determines whether the processing of steps S451 to S473 is completed for all tests related to the patient to be processed (S481).

When the processing of steps S451 to S473 is not completed for all the tests (S481: No), the display processing unit 103 returns the processing to step S451.

When the processing of steps S451 to S473 is completed for all the tests (S481: Yes), the display processing unit 103 executes the processing of step S491.

In step S491, the display processing unit 103 outputs a time series table, that is, test time-series information, to a screen of the terminal 5 according to a value stored in an array and setting of the background color. The test time-series information is displayed as the variant information time series field 523 in FIG. 9.

In steps S452, 462, and 472, the display processing unit 103 highlights a variant level and changes the degree of highlighting of the highlighted display according to a variant level.

### Expert Panel Support Screen 500

FIG. 9 is a diagram showing an example of the expert panel support screen 500.

The expert panel support screen 500 includes a disease information screen 510 and a variant information display screen 520.

The disease information screen 510 includes records of a "disease name", a "test date", a "test name", a "test type", and an "expert panel (EP) holding date". Further, the disease information screen 510 includes records of "classification", "prognosis", "treatment", and "other test".

These pieces of information are displayed based on the patient information 210, the test information 220, and the like shown in FIG. 3.

A record 511 of "prognosis" displays information on prognosis prediction based on a gene variant. Specifically, the record 511 of "prognosis" displays information based on "prognosis prediction" of the evaluation information 250 shown in FIG. 3.

The variant information display screen 520 displays a gene name field 521, a gene variant name field 522, and a variant information time series field 523. The variant information display screen 520 is displayed based on the gene variant information 240 shown in FIG. 3.

The gene name field 521 displays a name (standardized) of a gene in which a gene variant occurs. The gene name displayed in the gene name field 521 is the "gene name (standardized)" in the single nucleotide variant information 241, the copy number variant information 242, the structural variant information 243, and the other biomarker information 244 in FIG. 3.

A gene variant name is displayed in the gene variant name field 522. A name of a gene variant after GRCh38 conversion (that is, standardized) is displayed as the gene variant name. The gene variant name displayed in the gene variant name field 522 is the standard gene variant name obtained in steps S411, S421, and S431.

The variant information time series field 523 displays variant levels of a gene variant in time series. The variant level is a variant level in the gene variant. When a type of the gene variant is a single nucleotide variant (SNV), the variant level is an allele frequency. When a type of the gene variant is a copy number variant (CNV), the variant level is a copy number. When a type of the gene variant is a structural variant (SV), the variant level is a read number (expression level).

In the variant information time series field 523, the degree of highlighting of the background varies depending on a value of the variant level. Examples of the degree of highlighting include a density of the background color. In the example shown in FIG. 9, the density of the background color (that is, the degree of highlighting) is indicated by a density of dots. The density of the background color is set in FIG. 8B. Here, in addition to changing the density of the background color according to the value of the variant level, a method of changing the background color, changing a font size of the value of the variant level, or the like may be used.

Further, in the variant information time series field 523, a thinnest dot portion indicates that a corresponding gene variant is not a test target in a test corresponding to the portion. A field without dots (blank field) indicates that no corresponding gene variant is detected in a test.

The records of the variant information display screen 520 (the gene name field 521, the gene variant name field 522, and the variant information time series field 523) are displayed in the order sorted in the processing shown in FIG. 8A. That is, the variant information display screen 520 displays information based on a priority order related to a disease of a patient.

The star mark 524 indicates that a gene variant is a treatment target gene variant (gene variant serving as a treatment target). As described above, the star mark 524 is a gene variant to be focused, which is associated with a disease name of a patient in the focused variant information 300 shown in FIG. 4. The star mark 524 is displayed based on the flag added in the processing shown in FIG. 8B. By using the star mark 524, the gene variant serving as a treatment target is displayed on the terminal 5 in a visual expression different from other gene variants. Here, in addition to the star mark 524, other methods may be used, for example, a value of a variant level of the treatment target gene variant is indicated in bold, and a value of a variant level of a target gene variant is surrounded by a thick frame.

Although a single nucleotide variant, a copy number variant, and a structural variant are displayed in the example of the variant information time series field 523 shown in FIG. 9, other biomarkers may be displayed.

In the gene variant name, a gene variant to be focused is displayed in a higher rank according to a disease such as acute myeloid leukemia (AML), acute lymphocytic leukemia (ALL), and chronic myeloid leukemia (CML). That is, a variant level of a gene variant is displayed based on a priority order of a disease of a patient for a gene variant presented to a member. The priority order is the order sorted in the processing shown in FIG. 8A.

In the variant information time series field 523, gene variants detected in different tests are associated with one another in the gene name field 521 and the gene variant name field 522. For example, when a reference genome is different or a gene variant name is different depending on tests, the amino acid number may change depending on a difference in a standard transcriptome. Therefore, in the present embodiment, information is aligned with a standard variant position by standardizing a variant position and the like. Accordingly, gene variants detected in different tests can be associated with one another.

According to the present embodiment, the expert panel support screen 500 displays a change in a gene variant on the terminal 5 used by a member of an expert panel in a form visually recognizable by the member. Accordingly, it is easy for a user to visually recognize a change in a result of a cancer gene panel test.

According to the present embodiment, the display processing unit 103 highlights a variant level and changes the degree of highlighting (density of a background color in the example shown in FIG. 9) according to the variant level. Accordingly, a user can visually confirm a change in a result of a cancer gene panel test.

In the present embodiment, the records of the variant information display screen 520 (the gene name field 521, the gene variant name field 522, and the variant information time series field 523) shown in FIG. 9 are displayed based on a priority order related to a disease of a patient. In the present embodiment, the records of the variant information display screen 520 (the gene name field 521, the gene variant name field 522, and the variant information time series field 523) are displayed in the order sorted in the processing shown in FIG. 8A. Accordingly, a user can perform examination based on a priority order related to a disease of a patient.

Variant levels are displayed in time series in the variant information time series field 523 shown in FIG. 9. Accordingly, a plurality of test results and study results for the same case are displayed in time series. As a result, a user can easily grasp a time series change of the case, an optimal treatment can be easily studied, and the burden on the user is reduced.

Then, by using the star mark 524 shown in FIG. 9, the gene variant serving as the treatment target (treatment target gene variant) is displayed on the terminal 5 in a visual expression different from the other gene variants. Accordingly, it is easy for a user to visually recognize information on the treatment target gene variant.

Further, information of prognosis prediction based on a gene variant is displayed in the record 511 of "prognosis" shown in FIG. 9. Accordingly, it is easy for a user to visually recognize the prognosis prediction based on the gene variant and an effect of an actual treatment. The effect of an actual treatment is determined based on the time series variant levels of the gene variants displayed in the variant information time series field 523.

The invention is not limited to the embodiments described above and includes various modifications. For example, the embodiments described above have been described in detail in order to facilitate understanding of the invention, and the invention is not necessarily limited to those including all the configurations described above.

Although the terminal 5 is assumed to be a personal computer (PC) in the present embodiment, a smartphone, a tablet terminal, or the like may be used as the terminal 5.

Some or all of the above-described configurations, functions, units including the conversion unit 101 to the display processing unit 103, the registration information DB 2, the focused variant DB 3, the user related DB 4, and the like may be implemented by hardware, for example, by designing an integrated circuit. As shown in FIG. 2, the above-described configurations, functions, and the like may be implemented by software by a processor such as a CPU interpreting and executing a program for implementing each function. Information such as a program, a table, or a file that implements each function can be stored in a hard disk (HD), or can be stored in a recording device such as the memory 121 and a solid state drive (SSD), or a recording medium such as an integrated circuit (IC) card, a secure digital (SD) card, or a digital versatile disc (DVD).

Control lines and information lines considered to be necessary for description are shown in each embodiment, and not all control lines and information lines in a product are necessarily shown. Actually, it may be considered that almost all the configurations are connected to one another.

What is claimed is:

1. An expert panel support system for displaying a test result of a patient subjected to a gene panel test which is a target of an expert panel, the expert panel support system comprising:
an extraction unit configured to extract information related to a gene variant of the patient in a plurality of the gene panel tests; and
a display processing unit configured to display a change in the gene variant on a terminal used by a member of the expert panel in a form visually recognizable by the member.

2. The expert panel support system according to claim 1, comprising:
an acquisition unit configured to acquire a variant level of the gene variant for each gene panel test, wherein
the display processing unit highlights the variant level and changes a degree of the highlighting according to the variant level.

3. The expert panel support system according to claim 1, wherein
the display processing unit displays a variant level of the gene variant based on a priority order related to a disease of the patient for the gene variant to be presented to the member.

4. The expert panel support system according to claim 1, comprising:
a conversion unit configured to convert information that indicates substantially identical gene variants but is different between the gene panel tests into unified information by standardization.

5. The expert panel support system according to claim 1, wherein
a variant level of the gene variant is displayed in time series.

6. The expert panel support system according to claim 1, wherein
a gene variant serving as a treatment target is displayed on the terminal in a visual expression different from other gene variants.

7. The expert panel support system according to claim 1, wherein
the display processing unit displays prognosis prediction information based on the gene variant.

8. An expert panel support method comprising:
by an expert panel support system that displays a test result of a patient subjected to a gene panel test which is a target of an expert panel,
extracting information related to a gene variant of the patient in a plurality of the gene panel tests; and
displaying a change in the gene variant on a terminal used by a member of the expert panel in a form visually recognizable by the member.
